(19) United States
(12) Patent Application Publication
(10) Pub. No.: US 2023/0144045 A1
(43) Pub. Date: May 11, 2023

(54) LEAFLET ATTACHMENT MECHANISM AND STRAIGHT ATTACHMENT STRUTS

(71) Applicant: W. L. Gore & Associates, Inc., Newark, DE (US)

(72) Inventor: Dustin V. Dienno, Flagstaff, AZ (US)

(21) Appl. No.: 17/909,501

(22) PCT Filed: Mar. 2, 2021
(86) PCT No.: PCT/US2021/020455

§ 371 Date: Sep. 6, 2022

Related U.S. Application Data
(60) Provisional application No. 62/984,600, filed on Mar. 3, 2020.

(51) Int. Cl. A61F 2/24

(52) U.S. Cl. CPC A61F 2/2418; A61F 2250/0039; A61F 2250/0036; A61F 2250/0037; A61F 2230/0082

(57) ABSTRACT

A prosthetic valve including a leaflet frame configured to be transitioned from a collapsed state having a first diameter to an expanded state having a second diameter that is larger than the first diameter. The leaflet frame has an inlet and outlet end and defining a longitudinal axis. The leaflet frame includes a plurality of frame members that form a framework defining plurality of cells each having an interior cell space, the plurality of cells including an attachment cell. The plurality of frame members includes a plurality of sub-cell attachment members extending into the interior cell space of the attachment cell, the sub-cell attachment members each being configured to define a plurality of attachment locations along a sub-cell attachment line defined within a plane that intersects with the longitudinal axis such that the sub-cell attachment line extends through the interior cell space of the at least one attachment cell.

### CROSS-REFERENCE TO RELATED APPLICATION

This application is a national phase application of PCT Application No. PCT/US2021/020455, internationally filed on Mar. 2, 2021, which claims the benefit of Provisional Application No. 62/984,600, filed Mar. 3, 2020, which are incorporated herein by reference in their entireties for all purposes.

### FIELD

The present disclosure relates to implantable, expandable prosthetic devices and to methods and apparatuses for such prosthetic devices.

### BACKGROUND

The human heart can suffer from various valvular diseases. These valvular diseases can result in significant malfunctioning of the heart and ultimately require replacement of the native valve with an artificial valve. There are a number of known artificial valves and a number of known methods of implanting these artificial valves in humans. Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For example, collapsible transcatheter prosthetic heart valves can be crimped to a compressed state and percutaneously introduced in the compressed state on a catheter and expanded to a functional size at the desired position by balloon inflation or by utilization of a self-expanding frame or stent.

A prosthetic valve can be percutaneously introduced in a collapsed configuration on a catheter and expanded in the desired position by balloon inflation, mechanical expansion, or by utilization of a self-expanding support. A challenge in catheter-implanted prosthetic valves is control of perivalvular leakage around the valve, which can occur fora period of time following initial implantation. An additional challenge includes the process of crimping prosthetic valves to profiles suitable for percutaneous delivery to a patient.

### SUMMARY

Various disclosed concepts relate to prosthetic valves, and associated systems and methods, including a plurality of sub-cell attachment members configured to define a plurality of attachment locations along a sub-cell attachment line.

According to one example (“Example 1”), the prosthetic valve includes a leaflet frame configured to be transitioned from a collapsed state having a first diameter to an expanded state having a second diameter that is larger than the first diameter, the leaflet frame having an inlet end and an outlet end and defining a longitudinal axis, the leaflet frame including, a plurality of frame members that form a framework defining plurality of cells each having an interior cell space, the plurality of cells including at least one attachment cell, the plurality of frame members including a plurality of sub-cell attachment members extending into the interior cell space of the at least one attachment cell, the sub-cell attachment members each being configured to define a plurality of attachment locations along a sub-cell attachment line that is defined within a plane that intersects with, and is optionally transverse to, the longitudinal axis such that the sub-cell attachment line extends through the interior cell space of the at least one attachment cell.

According to another example further to Example 1 (“Example 2”), the prosthetic valve further includes a leaflet construct including a leaflet having a base attachment region, wherein the base attachment region of the leaflet is coupled to the leaflet frame along the sub-cell attachment line.

According to another example further to Examples 1 or 2 (“Example 3”), each of the plurality of sub-cell attachment members includes a projection base portion extending into the interior cell space and a projection head portion opposite the projection base portion, each of the projection head portions of the plurality of sub-cell attachment members being positioned along the sub-cell attachment line.

According to another example further to any of the preceding Examples (“Example 4”), the plurality of sub-cell attachment members includes a first sub-cell attachment member and a second sub-cell attachment member, and further wherein the at least one attachment cell has a top cell portion defined by the framework and a bottom cell portion defined by the framework, the first sub-cell attachment member extending from the top cell portion and the second sub-cell attachment member extending from the bottom cell portion.

According to another example further to Example 4 (“Example 5”), the first and second sub-cell attachment members are substantially parallel to each other when the leaflet frame is in the expanded state.

According to another example further to Examples 4 or 5 (“Example 6”), the top cell portion is defined by a first frame member and a second frame member of the plurality of frame members, and further wherein the first sub-cell attachment member includes a first leg extending from the first frame member and a second leg extending from second frame member, the first and second legs intersecting to form a first sub-cell attachment joint, the first sub-cell attachment joint being positioned adjacent the sub-cell attachment line when the leaflet frame is in the expanded state.

According to another example further to Examples 4 to 6 (“Example 7”), the bottom cell portion is defined by a third frame member and a fourth frame member of the plurality of frame members, and further wherein the second sub-cell attachment member includes a third leg extending from the third frame member and a fourth leg extending from the fourth frame member, the third and fourth legs intersecting to form a second sub-cell attachment joint, the second sub-cell attachment joint being positioned adjacent the sub-cell attachment line when the leaflet frame is in the expanded state.

According to another example further to Examples 4 to 7 (“Example 8”), the first sub-cell attachment member and the framework together define a shape resembling a parallelogram when the leaflet frame is in the expanded state, and optionally, wherein the second sub-cell attachment member and the framework combine to define a shape resembling a parallelogram when the leaflet frame is in the expanded state.

According to another example further to Examples 6 to 8 (“Example 9”), the first leg of the first sub-cell attachment member is shorter than the second leg of the first sub-cell attachment member, and optionally, wherein the first leg of the first sub-cell attachment member is thinner than the second leg of the first sub-cell attachment member.

According to another example further to Example 7 to 9 (“Example 10”), the third leg of the second sub-cell attachment member has substantially the same length and width as the first leg of the first sub-cell attachment member, and optionally, wherein the fourth leg of the second sub-cell attachment member has substantially the same length and width as the second leg of the first sub-cell attachment member.

According to another example further to any of the preceding Examples (“Example 11”), the plurality of frame members include a plurality of leaflet attachment members configured to be coupled to first and second sides of one or more leaflets of a leaflet construct, each of the plurality of leaflet attachment members having a first end portion at which each of the plurality of leaflet attachment members intersects with another one of the plurality of frame members, a second end portion at which each of the plurality of leaflet attachment members intersects with another one of the plurality of frame members, and an intermediate portion between the first and second end portions, the intermediate portion of each of the plurality of leaflet attachment members having a first curvature when the leaflet frame is in the collapsed state and a second curvature when the leaflet frame is in the expanded state, the first curvature being greater than the second curvature.

According to another example further to Example 11 (“Example 12”), the first curvature is defined as a maximum transverse deviation of the intermediate portion from a straight line drawn between the first and second end portions, the maximum transverse deviation having a value of 0.5 (1/mm).

According to another example further to Examples 11 or 12 (“Example 13”), the second curvature is defined as a maximum transverse deviation of the intermediate portion from a straight line drawn between the first and second end portions, the maximum transverse deviation having a value of 0.05 (1/mm).

According to another example further to Examples 11 to 13 (“Example 14”), the second curvature is at least 20% less than the first curvature.

According to another example further to Examples 11 to 14 (“Example 15”), the intermediate portions of at least two of the leaflet attachment members are linearly aligned with one another when the leaflet frame is in the expanded state.

According to another example further to Examples 11 to 15 (“Example 16”), the intermediate portions of each of the leaflet attachment members has a recurved shape when the leaflet frame is in the collapsed state.

According to another example further to Examples 11 to 16 (“Example 17”), the prosthetic valve further includes a leaflet construct including a leaflet having a side attachment region, the side attachment region being coupled to at least one of the plurality of leaflet attachment members, and optionally, wherein the side attachment region is coupled to at least two of the plurality of leaflet attachment members having intermediate portions that are in linear alignment when the leaflet frame is in the expanded state.

According to one example (“Example 18”), a prosthetic valve includes a leaflet frame configured to be transitioned from a collapsed state having a first diameter to an expanded state having a second diameter that is larger than the first diameter, the leaflet frame having an inlet end and an outlet end and defining a longitudinal axis, the leaflet frame including, a plurality of frame members defining a framework forming a pattern of cells, the plurality of frame members including a plurality of leaflet attachment members configured to be coupled to first and second sides of one or more leaflets of a leaflet construct, each of the plurality of leaflet attachment members having a first end portion at which each of the plurality of leaflet attachment members intersects with another one of the plurality of frame members, a second end portion at which each of the plurality of leaflet attachment members intersects with another one of the plurality of frame members, and an intermediate portion between the first and second end portions, the intermediate portion of each of the plurality of leaflet attachment members having a first curvature when the leaflet frame is in the collapsed state and a second curvature when the leaflet frame is in the expanded state, the first curvature being greater than the second curvature.

According to another example further to Example 18 (“Example 19”), the first curvature is defined as a maximum transverse deviation of the intermediate portion from a straight line drawn between the first and second end portions, the maximum transverse deviation having a value of 0.5 (1/mm).

According to another example further to Examples 18 or 19 (“Example 20”), the second curvature is defined as a maximum transverse deviation of the intermediate portion from a straight line drawn between the first and second end portions, the maximum transverse deviation having a value of 0.05 (1/mm).

According to another example further to Examples 18 to 20 (“Example 21”), the second curvature is at least 20% less than the first curvature.

According to another example further to Examples 18 to 21 (“Example 22”), the intermediate portions of at least two of the leaflet attachment members are linearly aligned with one another when the leaflet frame is in the expanded state.

According to another example further to Examples 18 to 22 (“Example 23”), the intermediate portions of each of the leaflet attachment members has a recurved shape when the leaflet frame is in the collapsed state.

According to another example further to Examples 18 to 23 (“Example 24”), the prosthetic valve further includes a leaflet construct including a leaflet having a side attachment region, the side attachment region being coupled to at least one of the plurality of leaflet attachment members, and optionally, wherein the side attachment region is coupled to at least two of the plurality of leaflet attachment members having intermediate portions that are in linear alignment when the leaflet frame is in the expanded state.

According to one example (“Example 25”), a method for treating a human patient with a diagnosed condition or disease associated with valve insufficiency or valve failure of a native valve is provided, the method comprising implanting the prosthetic valve of any of preceding Examples, the prosthetic valve being implanted at or adjacent to a location associated with the native valve.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a prosthetic valve being delivered via a catheter according to an embodiment disclosed herein;

FIG. 2 shows a section of a prosthetic valve according to an embodiment disclosed herein;

FIG. 3 shows the section of the prosthetic valve of FIG. 2 in an expanded state;

FIG. 4 shows a close-up view of the section the prosthetic valve of FIG. 2 in a compressed state;

FIG. 5 shows a section of a prosthetic valve in an expanded state according to an embodiment disclosed herein;

FIG. 6 shows a section of a prosthetic valve according to an embodiment disclosed herein;

FIG. 7 shows the section of the prosthetic valve of FIG. 6 in an expanded state;

FIG. 8 shows a prosthetic valve in an expanded state according to an embodiment disclosed herein;

FIG. 9 shows a section of a prosthetic valve in a compressed state according to an embodiment disclosed herein;

FIG. 10 shows a section of a prosthetic valve according an embodiment disclosed herein;

FIG. 11 shows the section of the prosthetic valve of FIG. 10 in an expanded state;

FIG. 12 shows frame members in two different configurations according to an embodiment disclosed herein;

FIG. 13 shows a prosthetic valve according to an embodiment disclosed herein;

FIG. 14 shows a prosthetic valve with straight frame members for the leaflet side attachment region and sub-cell attachment members for the leaflet base attachment region according to an embodiment disclosed herein;

FIG. 15 shows a leaflet according to an embodiment disclosed herein;

FIG. 16 shows the leaflet of FIG. 15 coupled with a frame projection portion of a frame member according to an embodiment disclosed herein.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to the surface characteristics, a “developable surface” is defined as a surface with zero (0) Gaussian curvature so the surface can be flattened onto a plane without distortion such as stretching or compressing. For example, when a sphere is flattened on a plane, similar to how a globe is to be flattened to form a world map, there is always a distortion of the surface because the surface of the sphere has a positive non-zero Gaussian curvature of 1/r² throughout, where r is the radius of the sphere. In another example, the surface of a hyperboloid has a negative non-zero Gaussian curvature. However, when a cylinder or cone is flattened onto a plane, there is no distortion on the surface of the cylinder or cone because these shapes can be formed by cutting and developing a sheet of material. As such, examples of developable surfaces include cylinders and cones, as well as oloids and sphericons.

With respect to terminology of inexactitude, the terms “about”, “substantially” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example.

With respect to relative terminology, such as up, down, vertical, horizontal and the like, the referenced components may be oriented in any direction. Similarly, throughout this disclosure, where a process or method is shown or described, the method may be performed in any order or simultaneously, unless it is clear from the context that the method depends on certain actions being performed first.

The term “leaflet” or “leaflet construct” which comprises a plurality of leaflets as used herein in the context of prosthetic valves is a component of a one-way valve wherein the leaflet is operable to move between an open and closed position under the influence of a pressure differential. In an open position, the leaflet allows fluid (e.g., blood) to flow through the valve. In a closed position, the leaflet substantially blocks retrograde flow through the valve by occluding the valve orifice. In embodiments comprising multiple leaflets, each leaflet cooperates with at least one neighboring leaflet or secondary structure to block the retrograde flow of blood. The pressure differential in the blood is caused, for example, by the contraction of a ventricle or atrium of the heart, such pressure differential typically resulting from a fluid pressure building up on one side of the leaflets when closed, for example, by the contraction of a ventricle or atrium of the heart. As the pressure on an inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve rises above the blood pressure on the inflow side of the valve, the leaflet returns to the closed position generally preventing retrograde flow of blood through the valve.

It is appreciated that leaflets, where not required by the specific design or mode of function of the disclosed embodiment, may be rigid such as in mechanical valves or may be flexible as in bioprosthetic and synthetic valves. It is further appreciated that, although embodiments provided herein include a frame that supports the leaflets, the leaflets may not necessarily be supported by a frame. In other embodiments, the leaflets may be constructed as in the tissue valve art that are formed into the desired shape without a frame.

The term “membrane” as used herein refers to a sheet of material comprising a single composition, such as, but not limited to, expanded fluoropolymer.

The term “composite material” as used herein refers to a combination of a membrane, such as, but not limited to, expanded fluoropolymer, and a polymer. The polymer may be an elastomer, elastomeric, or non-elastomeric material, such as, but not limited to, a fluoroelastomer. The elastomer, elastomeric or non-elastomeric material can be imbibed within a porous structure of the membrane, coated on one or both sides of the membrane, or a combination of coated on and imbibed within the membrane.

As used herein, the term “elastomer” refers to a polymer or a mixture of polymers that has the ability to be stretched to at least 1.3 times its original length and to retract rapidly to approximately its original length when released. The term “elastomeric material” refers to a polymer or a mixture of polymers that displays stretch and recovery properties similar to an elastomer, although not necessarily to the same degree of stretch and/or recovery. The term “non-elastomeric material” refers to a polymer or a mixture of polymers that displays stretch and recovery properties not similar to either an elastomer or elastomeric material, that is, considered not an elastomer or elastomeric material as is generally known.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

The term “frame” as used herein generically refers to any structure or support used to directly or indirectly support leaflets for use in the prosthetic valve. It will be understood that, where appropriate, that the term frame may be used interchangeably with support structure. In accordance with some embodiments, the leaflets may be supported by the wall of a solid-walled conduit, the solid-walled conduit being understood to be a frame or support structure.

### Description of Various Embodiments

Prosthetic valves have been developed to replace a native valve and may include leaflets that are flexible and a support structure to which the leaflets are coupled. Leaflets may be fabricated from biological tissue, synthetic materials, or combinations thereof. In some valve designs, the support structure includes a relatively rigid frame to which the leaflets are coupled to support the leaflets and provide dimensional stability to the leaflets after the prosthetic valve is implanted. In operation, the leaflets move under the influence of fluid pressure with the leaflets moving toward an open configuration when the upstream fluid pressure exceeds the downstream fluid pressure and moving toward a closed configuration when the downstream fluid pressure exceeds the upstream fluid pressure. In the closed configuration, the leaflets coapt with an adjacent leaflet, the support structure, or combinations thereof under the influence of the downstream fluid pressure. In this manner, downstream pressure closes the valve by pushing against the leaflets of the closed valve to prevent downstream blood from flowing retrograde through the valve.

In embodiments comprising multiple leaflets, each leaflet generally cooperates with at least one neighboring or adjacently situated leaflet or structure to block or restrict the retrograde flow of blood. The pressure differential in the blood is caused, for example, by the contraction of a ventricle or atrium of the heart, such pressure differential typically resulting from a fluid pressure building up on one side of the leaflets when closed. As the pressure on the inflow side of the valve rises above the pressure on the outflow side of the valve, the leaflets open and blood flows therethrough. As blood flows through the valve into a neighboring chamber or blood vessel, the pressure on the inflow side equalizes with the pressure on the outflow side. As the pressure on the outflow side of the valve raises above the blood pressure on the inflow side of the valve, the leaflet returns to the closed position generally preventing retrograde flow of blood through the valve.

Embodiments herein include various apparatuses, systems, and methods for a prosthetic valve suitable for surgical and/or transcatheter placement, such as, but not limited to, cardiac valve replacement. The valve is operable as a one-way valve wherein the valve defines a valve lumen which permits flow when the leaflets are in an open position and which may be occluded when the leaflets are in a closed position and thus prevent flow through the valve lumen, wherein the leaflets are in open and closed positions in response to differential fluid pressure.

The following non-limiting examples are provided to further illustrate embodiments. It should also be readily appreciated that other valve frame designs may be used other than those illustrated in the examples below and accompanying figures.

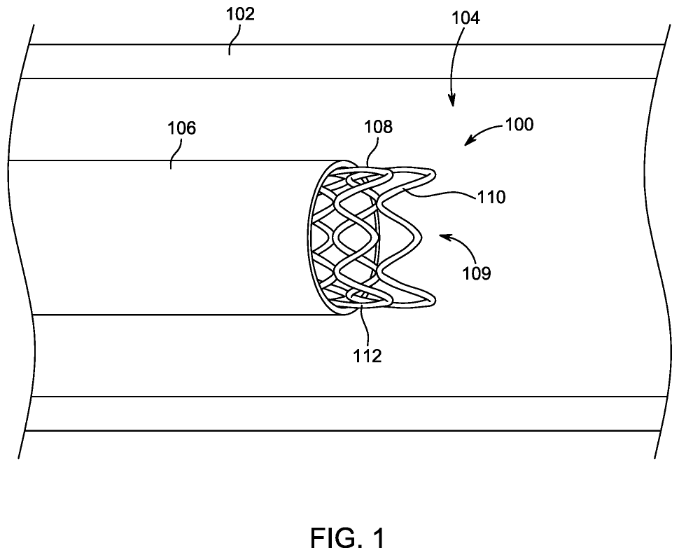

FIG. 1 shows a prosthetic valve 100 according to some embodiments. In some embodiments, the prosthetic valve 100 is delivered into a vessel 102, for example a blood vessel within a body, such that the prosthetic valve 100 can be enclosed inside a lumen 104 of the vessel 102. A delivery catheter 106 can be used to maintain the prosthetic valve 100 in a collapsed or compressed state before the prosthetic valve 100 is deployed inside the lumen 104. As the prosthetic valve 100 is deployed, the prosthetic valve 100 takes on a more expanded state in which the outer periphery, or diametric profile, of the prosthetic valve 100 is greater than the outer periphery, or diametric profile, in the compressed state. In some examples, the prosthetic valve 100 includes a substantially circular cross-section, therefore the diameter of the prosthetic valve 100 increases when deployed from the delivery catheter 106.

The prosthetic valve 100 includes a leaflet frame 108 formed by a framework 109 defined by a plurality of frame members 110. As shown in FIG. 1, the frame members 110 define a plurality of cells 112. The cells 112 defined by the frame members 110 define a surface area, wherein the surface area is variable as the prosthetic valve 100 expands from a compressed configuration. When the prosthetic valve 100 is deployed, the frame members 110 can deform such that the leaflet frame 108 expands to define a generally cylindrical shape. The leaflet frame 108 need not define a right-cylindrical shape, but may have a tapered, cylindrical shape in the expand configuration, compressed configuration, or both. For example, the frame of a 26-mm prosthetic valve, when crimped, has a first diameter of 14 French at the outflow end of the prosthetic valve and a second diameter of 12 French at the inflow end of the prosthetic valve.

The leaflet frame 108 can be made of any of various suitable plastically-expandable materials (e.g., stainless steel, etc.) or self-expanding materials (e.g., nickel titanium alloy (NiTi), such as nitinol). When constructed of a plastically-expandable material, the leaflet frame 108, and thus the prosthetic valve 100, can be compressed to a radially collapsed configuration in the delivery catheter 106 and then expanded inside a patient by an inflatable balloon or equivalent expansion mechanism (not shown). When constructed of a self-expandable material, the leaflet frame 108, and thus the prosthetic valve 100, can be crimped or otherwise transitioned to a radially collapsed configuration and restrained in the collapsed configuration by insertion into a sheath, fiber constraint, and/or other mechanisms of the delivery catheter 106. The prosthetic valve 100 can be advanced from the delivery sheath, which allows the prosthetic valve to expand to its functional size.

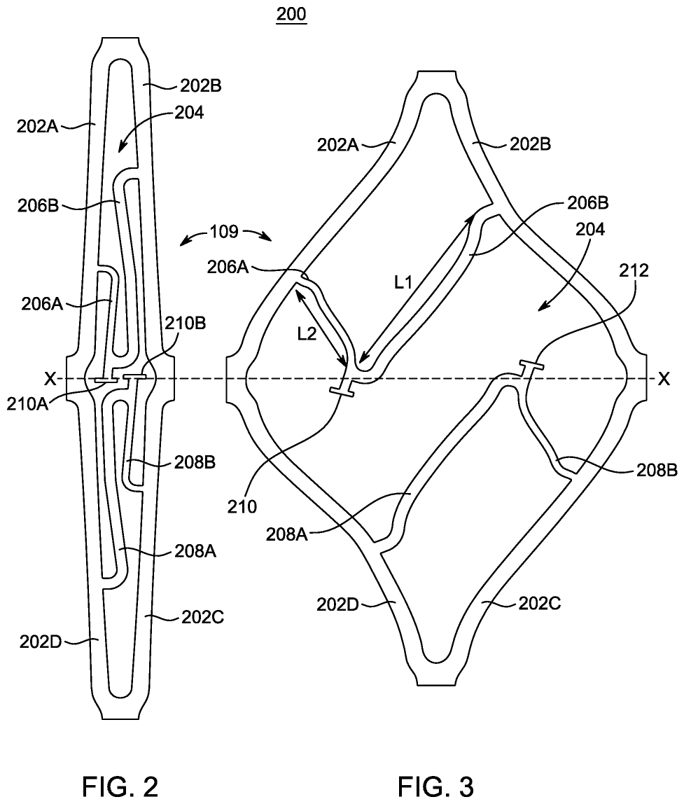

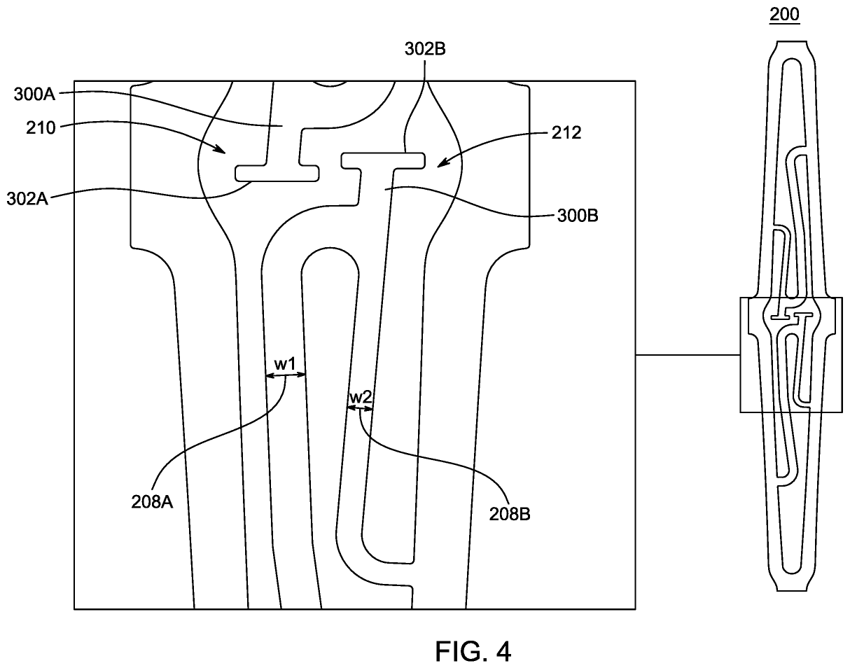

FIGS. 2 to 4 show a section, or an attachment cell 200, of a prosthetic valve according to some embodiments, such as the prosthetic valve 100 in FIG. 1. FIG. 2 shows the attachment cell 200 in a compressed state, FIG. 3 shows the attachment cell 200 in an expanded state, and FIG. 4 shows a close-up view of the attachment cell 200. The framework 109 defines a plurality of such cells or similar cells positioned adjacent each other to form the framework 109. Each cell is defined by a plurality of frame members 202, where each of the frame members 202 comprise at least a portion of the leaflet frame 108. The attachment cell 200, according to some embodiments, is defined by four frame members 202A, 202B, 202C, and 202D as shown.

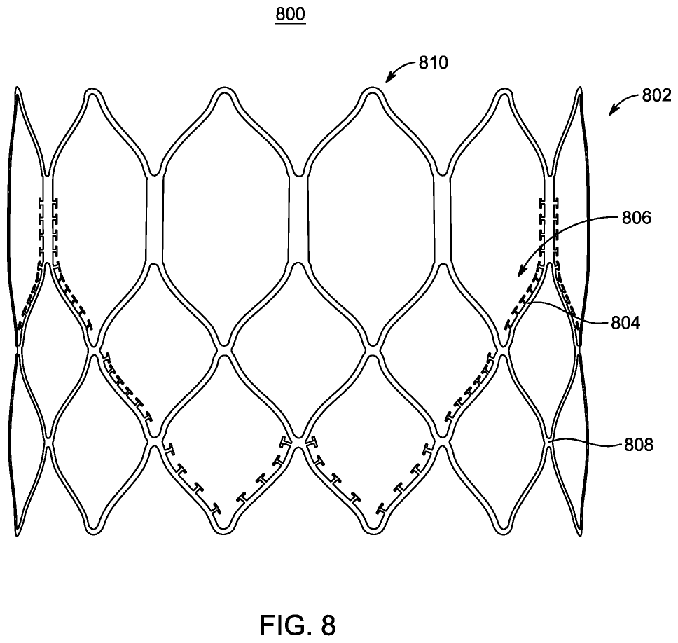

The frame members 202 collectively form the framework 109 that defines the attachment cells 200 or a pattern of cells, each cell having an interior cell space 204. Referring to FIG. 3, the attachment cell 200 is in a shape resembling a diamond or rhombus when in the open state. The attachment cell 200 also has a set of sub-cell attachment members 206 and 208 extending from the frame members 202 toward the interior cell space 204. In some examples, the set of sub-cell attachment members 206 may include two or more sub-cell attachment members. In at least one example, the set includes two sub-cell attachment members 206 and 208 where each attachment member has two legs: a first leg 206A, 208A with a length of L1 and a second leg 206B, 208B with a length of L2 that is less than L1. In some examples, two of the frame members 202 and the sub-cell attachment members 206 or 208 (e.g., the first and second frame members 202A, 202B and first sub-cell attachment member 206) that extend therefrom together form a shape resembling a parallelogram when the framework 109 is expanded. That is, in the expanded state, the legs of sub-cell attachment member 206 or 208 are substantially parallel with the frame member 202 to which they are not directly attached (e.g., when the first leg 206A extends from the first fame member 202A and the second leg 206B extends from the second frame member 202B, the first leg 206A is substantially parallel with second frame member 202B and the second leg 206B is substantially parallel with first frame member 202A). In some embodiments, the frame members 202 may be wider than the sub-cell attachment members 206 and 208. The sub-cell attachment members 206 and 208 may extend from or connect to the frame members 202 at an intermediate portion of the frame members 202. Where the frame members 202 form the framework 109 of the leaflet frame 108, some of the frame members 202 intersect or connect at connector portions 808, shown in FIG. 8, while other frame members 202 form an apex 810.

Referring still to FIGS. 2-4, a first frame projection portion 210 extends from the first sub-cell attachment member 206, and a second frame projection portion 212 extends from the second sub-cell attachment member 208. For example, the frame projection portion 210 includes a projection base portion 300A and a projection head portion 302A opposite the projection base portion 300A, and the frame projection portion 212 includes a projection base portion 300B and a projection head portion 302B opposite the projection base portion 300B. In some examples, the projection head portion 302 extends transverse with respect to the projection base portion 300. In some examples, the projection head portion 302 extends linearly from the projection base portion 300. Furthermore, in some examples, the width of the longer leg 206A, 208A (shown with “W1” in FIG. 4) is greater than the width of the shorter leg 206B, 208B (shown with “W2” in FIG. 4).

In some examples, the ratio of the lengths L1, L2 and the ratio of the widths W1, W2 are substantially equal (e.g., W1/W2=L1/L2). That is, the ratio of the longer length L1 to the shorter length L2 is the same as the ratio of the wider width W1 to the narrower width W2. Keeping the ratio constant between the length and the widths can help equalize the length differential and the bending stiffness differential between various lengths and widths of the opposing frame projection portions 210, 212. In some examples, the bending stiffness of the members is similar to allow the sub-structure to deform in a shape as close to parallelogram as possible to control the position of the attachment point in the expanded version.

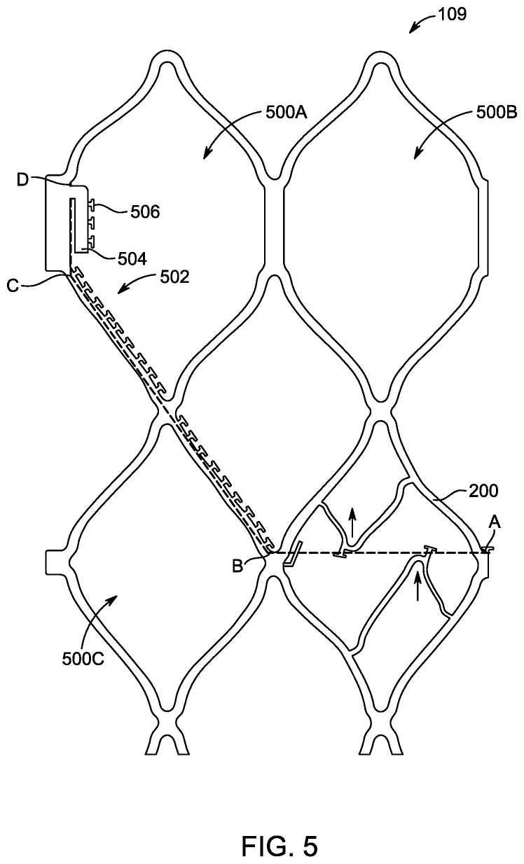

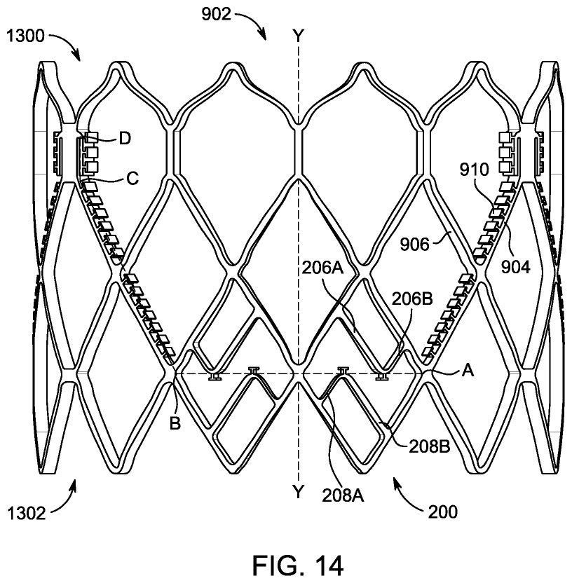

FIGS. 2 and 3 also show a dotted line (X-X) representing the position of an imaginary plane extending through the prosthetic valve 100. As seen in the figures, the line X-X extends across a portion of the attachment cell 200 and represents a potential location of where a leaflet (not shown) may be attached to the framework 109 formed by the frame members 202. The line X-X may be described as a plane in order to represent the position of portions of the attachment line relative to the prosthetic valve 100 when the prosthetic valve 100 is shown in either a flat configuration as seen in at least FIG. 5 or in a cylindrical (or any other applicable) configuration as seen in FIG. 14. Thus, the plane is described for the purposes of frame of reference and describing the position of an attachment line. With reference to the attachment line, one example provides that the sub-cell attachment members 206, 208 extend into the interior cell space 204 of an attachment cell 200 such that the sub-cell attachment members 206, 208 define a plurality of attachment locations along a sub-cell attachment line. Because the sub-cell attachment members 206, 208 extend into the interior cell space 204 of the attachment cell 200, the sub-cell attachment line also extends into or through the interior cell space 204 of the attachment cell 200.

In some embodiments, the plane may be disposed relative to the prosthetic valve 100 such that the plane intersects the longitudinal axis defined in the prosthetic valve 100, and consequently the prosthetic valve 100 itself. In some embodiments, the plane is perpendicular or transverse to the longitudinal axis. In some embodiments, an attachment line or sub-cell attachment line is positioned to extend into or through the interior cell space 204 of at least one of the cells of the prosthetic valve 100. In some embodiments, the sub-cell attachment line is defined along the line X-X which represents an intersection of the plane with the prosthetic valve 100. Thus a leaflet construct may be coupled to the leaflet frame 108 at least partially at the sub-cell attachment line.

As presented herein, examples of prosthetic valves include flexible leaflets that move in response to changing fluid pressure. The leaflets may be provided as individual components, referred to as leaflets, or may be part of a larger multi-leaflet component referred to as a leaflet construct. It is appreciated that leaflets and leaflet constructs may be constructed in many ways. By way of example, a leaflet may be cut out of a flat sheet or tubular member. By way of another example, a leaflet construct may be cut out of a tubular member or a flat sheet that has been subsequently rolled into a tubular shape. These are but a few examples of forming leaflets or leaflet constructs, which also include, but not limited to, compression and injection molding processes.

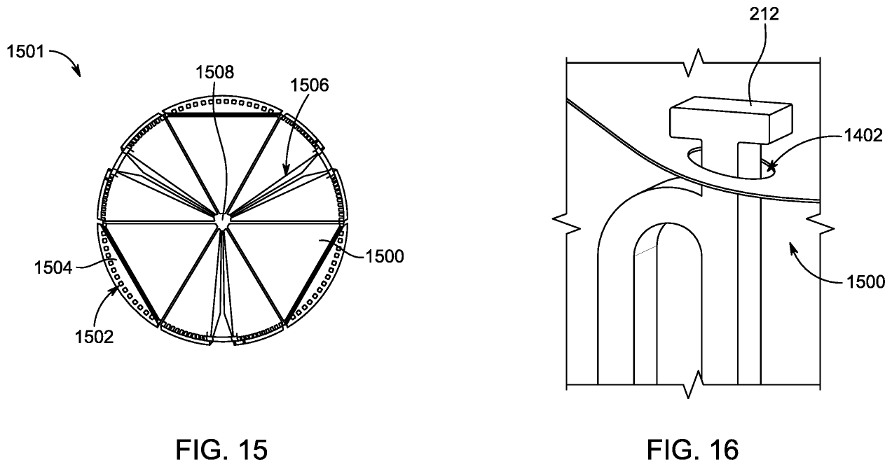

For example, FIG. 15 shows a leaflet construct 1501 with one or more leaflets 1500, according to some embodiments in which each leaflet 1500 has a plurality of openings 1502 or apertures on the surface proximate an outer periphery of each of the leaflets 1500, including a portion called a base attachment region 1504, wherein the base attachment region 1504 of each of the leaflets 1500 is coupled to the leaflet frame 108 along a sub-cell attachment line. The frame projection portion 210, 212 extends through the openings 1502 such that the frame projection portion 210, 212 restricts the movement of the leaflet 1500 and prevents the base attachment region 1504 of each of the leaflet 1500 from sliding off the frame projection portion 210, 212 as shown in FIG. 15. In some examples, the frame projection portions 210, 212 are equally spaced apart along the line X-X that extends through the attachment cell 200 to provide stable support for each of the leaflets 1500. In some examples, the frame projection portions 210, 212 also serve the purpose of preventing the respective leaflets 1500 from distorting during compression of the prosthetic valve 100 or have non-ideal shape when deployed from the delivery catheter 106. In some examples, the frame projection portions 210 or 212 apply radial force of the respective leaflets 1500 toward a longitudinal axis of the prosthetic valve 100.

FIG. 5 is a close-up of a portion of the framework 109 illustrating example attachment features of the prosthetic valve 100 for a portion of a leaflet 1500 (e.g., a partial leaflet attachment window is provided in FIG. 5). An example of a full leaflet attachment window fora leaflet 1500 on a prosthetic valve may be seen in at least FIG. 14. Although FIG. 5 is specifically referenced herein, many of the features of the framework 109 shown in FIG. 5 are likewise shown in FIG. 14. Referring to FIG. 5, the framework 109 may include one or more cells without sub-cell attachment members 206, 208, such as cells 500A, 500B, 500C and one or more cells with sub-cell attachment members 206, 208. In the example shown, the cells 500A, 500B, and 500C have no sub-cell attachment members; however, in some embodiments the cells 500A, 500B, and/or 500C include such attachment members as appropriate. One or all of the one or more leaflets 1500 may be disposed along attachment lines, such as the dotted line shown in FIG. 5 that extends from point A to point B, and which crosses proximate a center portion of the attachment cell 200, then from point B to point C along the side of two cells 500A and 500C, and lastly from point C toward and ending at point D in the cell 500A. Thus, line A-B may represent the sub-cell attachment line, in this case a base attachment line, and lines B-C and C-D represent other attachment lines, for example, side and commissure attachment lines. The cells 500A and 500C include a plurality of leaflet frame projections 502 along the frame members that define the framework 109 of the prosthetic valve 100 in a substantially straight configuration, as shown by the line B-C. Each of the leaflet frame projections 502 extends through one of the openings 1502 of the leaflet 1500 to restrict movement of the leaflet 1500. In some examples, the cell 500A also includes an additional sub-cell attachment member 504 extending from the framework 109 to provide additional leaflet attachment members 506 on the surface.

In various embodiments, adjacently situated leaflet frame projections 502 can be spaced apart from each other a distance that sufficiently disperses the load on the base attachment region 1504 of the leaflet 1500, as shown in FIG. 14, without significantly affecting the structural integrity thereof. In addition, the distance can be adjusted for the base attachment region 1504 to abut sufficiently with the leaflet frame projections 502. Various non-limiting example distances are within a range of between about 0.5 mm to about 2 mm, such as about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm, for example, or any range between any of the foregoing.

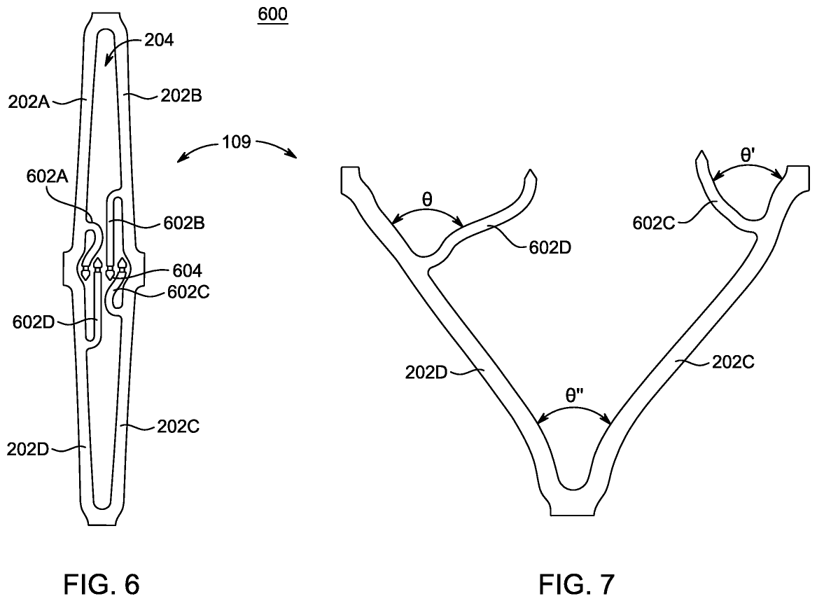

FIGS. 6 and 7 show another example of a section, or cell 600, of a prosthetic valve according to some embodiments, such as the prosthetic valve 100 in FIG. 1. More specifically, FIG. 6 shows the cell 600 in a compressed state and FIG. 7 shows a portion, or more specifically the bottom portion, of the cell 600 in an expanded state. The cell 600 includes four frame members 202A, 202B, 202C, and 202D, similar to the attachment cell 200 in FIGS. 2 to 4. The cell 600 further has a sub-cell attachment member 602 extending from each frame member such that the first sub-cell attachment member 602A extends from the first frame member 202A, the second sub-cell attachment member 602B extends from the second frame member 202B, the third sub-cell attachment member 602C extends from the third frame member 202C, and the fourth sub-cell attachment member 602D extends from the fourth frame member 202D. The sub-cell attachment members 602 resemble a set of cantilevers protruding from the frame members 202 in an offset configuration so that each sub-cell attachment member 602 does not interrupt movement of the other sub-cell attachment members 602.

Each sub-cell attachment member 602 is independent from the other sub-cell attachment members 602 so that the sub-cell attachment members 602 are spaced apart and do not come into contact with each other in the expanded state. In some examples, the sub-cell attachment members 602 also include frame projection portions 604 which may resemble various shapes to restrict movement of the leaflet 1500 which attaches to the frame projection portions 604. In some examples, the frame projection portions 604 may have a spade-like configuration, a T-shaped configuration, a hooked configuration, a pointed configuration, or any other suitably shaped configuration.

When the cell 600 is expanded to the configuration shown in FIG. 7, the angle formed between the sub-cell attachment members 602 and the respective frame members 202 from which the sub-cell attachment members 602 extend increases along with the angle between two neighboring frame members 202 in the upper and lower corners of the cell 600. This angle is represented in FIG. 7 with e, e′, and e″ as shown. In some examples, the angles e, e′, and e″ are all different but are greater than a predetermined angle. This is because, as the framework 109 expands from the compressed state of FIG. 6 to the expanded state of FIG. 7, the angle between each sub-cell attachment member 602 and its respective frame member 202, the angle between the frame members 202A and 202B (not shown in FIG. 7), and the angle between the frame members 202C and 202D all increase, such that all of the aforementioned angles are greater in the expanded state than in the compressed state.

FIG. 8 shows a framework 800 of a prosthetic valve 802 as seen from the side, where the framework 800 includes a plurality of frame members 804 and a plurality of frame projection portions 806 extending from the frame members 804. The frame projection portions 806 can extend along a side of the frame members 804 so as to form a curved configuration when the prosthetic valve 802 is expanded. In some examples, the curved configuration is recurved, serpentine, or S-shaped in the longitudinal direction.

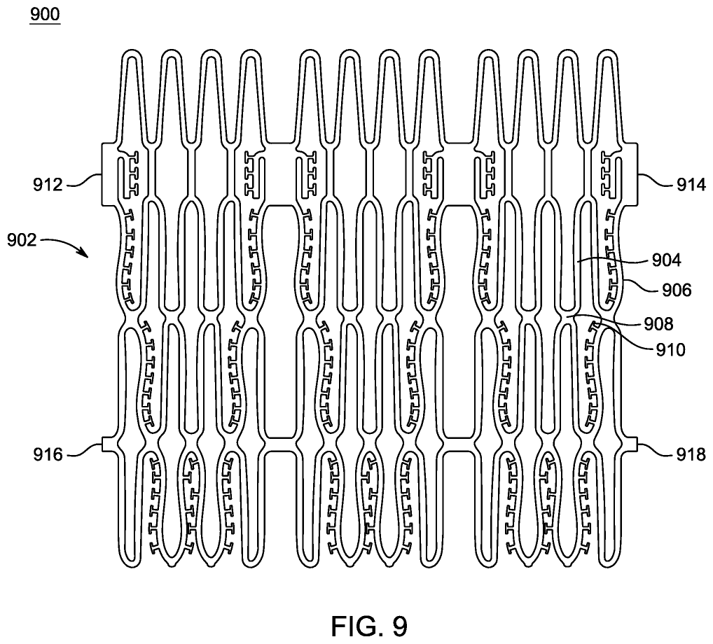

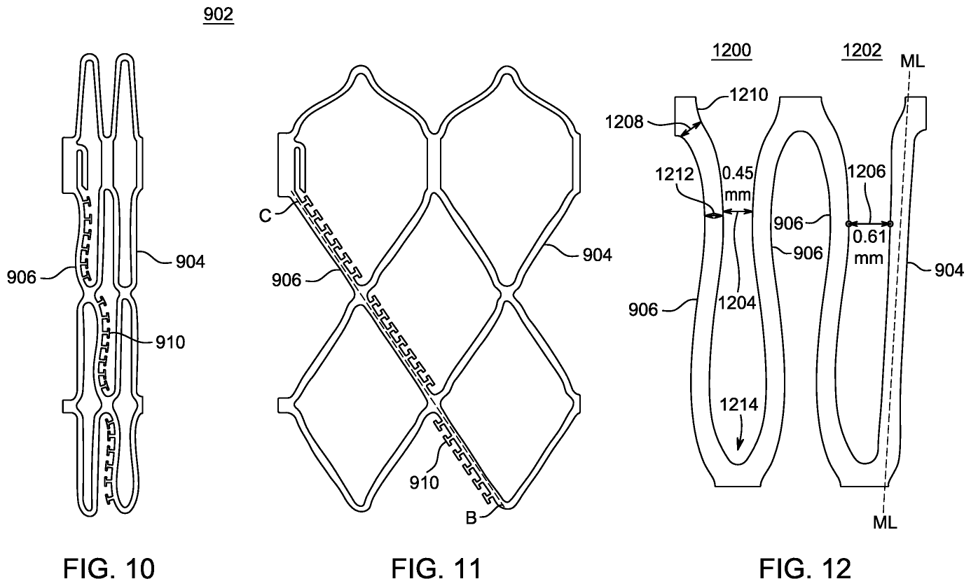

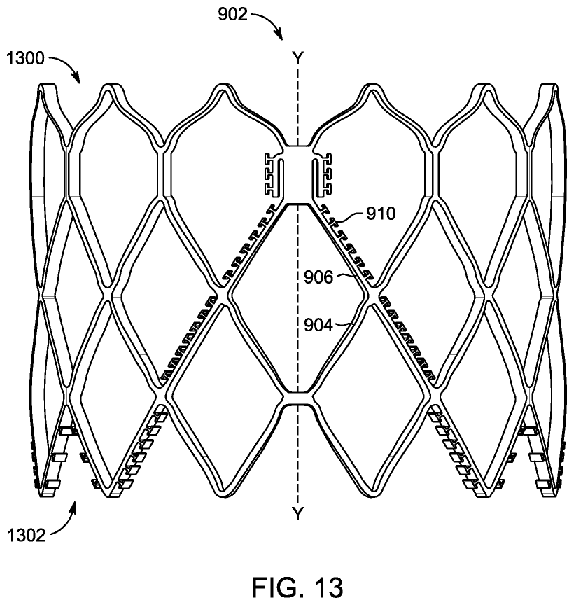

FIG. 9 shows a sheet of material (for example, steel or nitinol) that is cut to form a pattern 900, which is then developed into a leaflet frame of a prosthetic valve. As such, the pattern 900 defines the shape of a framework 902 that forms the leaflet frame. As can be seen in FIGS. 9-13, the pattern 900 may be configured in an unexpanded configuration 900A and an expanded configuration 900B. In some examples, the pattern 900 may be in the unexpanded configuration 900A, as seen in FIG. 10, and is made such that the framework 902 includes first frame members 904 that are substantially straight and second frame members 906 that are substantially curved. The sheet of material is shown in an as-cut, crimped state where the first frame members 904 are substantially straight in the crimped state and can become substantially curved in the expanded state; furthermore, the second frame members 906 are substantially curved in the crimped state and can become substantially straight in the expanded state, when appropriately designed. In some examples, all the frame members 904, 906 are either substantially straight or substantially curved. Each frame member 904, 906 that is straight either in the crimped or expanded state may have a curved portion near an intersection 908 between the frame member 904, 906 and a neighboring frame member but is otherwise substantially straight. For reference, a frame member that is substantially straight includes a frame member having a “straight portion” in which a curvature of a midline (shown by a line “ML” in FIG. 12) of the straight frame member, defined as the line parallel to and in the middle of the two opposing edges at the center of the straight frame member, is less than 0.05 mm⁻¹ (may also be notated as 1/mm).

Each frame member 904, 906 that is curved along its length may include a curved, recurved, serpentine, or S-shaped portion in the framework 902 when initially cut out from the sheet of material. In some examples, the frame member 904, 906 when curved has a curvature of between 0.05 and 0.1 mm⁻¹, between 0.1 and 0.2 mm⁻¹, between 0.2 and 0.3 mm⁻¹, between 0.3 and 0.4 mm⁻¹, between 0.4 and 0.5 mm⁻¹, or greater than 0.5 mm⁻¹ along the curved portion. It will be understood that in some embodiments, when the frame member 904, 906 is straight, the frame member 904, 906 may have a curvature that is less than or equal to 20%, less than or equal to 10%, or less than or equal to 5% of the curvature of a frame member 904, 906 that is straight. For example, a first frame member 904 that is curved may have a curvature of 0.50 mm^{'1} along a curved portion, whereas a second frame member 906 that is straight may have a curvature of 0.05 mm⁻¹ along a length or portion of the second frame member 906. It will be understood that the curvature can be measured relative to either the midline ML or can be measured along the surface of the frame member 904, 906 (e.g., along the surface from which the frame projection portions 910 extend). In some examples, the framework 902 can have a maximum curvature or a mean curvature in the aforementioned range. In such a case, the mean curvature is determined from averaging the curvatures of all the frame members that are curved in the framework 902.

In some examples, a plurality of frame projection portions 910 extend from the frame members 904, 906 that are curved. In some examples, the frame projection portions 910 can extend from the frame members 904, 906 that are straight. Generally, having frame members 904, 906 that are curved increases the packing density of the pattern 900 compared to straight frame members. The leaflet frames discussed herein are formed by cutting a pattern from a tube of material, for example a material in a cylindrical configuration, and therefore are developable surfaces.

In some examples, after the pattern 900 is laser-cut from a sheet of material to form the framework 902, the pattern 900 is then rolled into a cylinder such that at least some, if not most or all of, the frame members 904, 906 that are straight in the crimped configuration are substantially parallel with respect to a longitudinal axis of the resulting cylinder. After the pattern 900 is rolled into the cylinder, two ends of the framework 902 are attached to each other. For example, in FIG. 9, a first end portion 912 is attached to a second end portion 914, and a third end portion 916 is attached to a fourth end portion 918. The means of attachment include gluing, bonding, welding, and the like. Furthermore, in some examples, each of the end portions 912, 914, 916, 918 includes a hook or some kind of attachment mechanism that can lock into one another to attach the respective end portions together. In some examples, one end portion can have a hook and the other end portion can have an opening through which the hook can be inserted. The leaflet 1500 is attached to the frame projection portions 910 to form a prosthetic valve 100.

In some examples, the pattern 900 is laser-cut from a tube that defines the cylindrical shape of the framework 902. The tube has a similar circumference as a width of the pattern defined by the framework 902 in the compressed state. The laser-cut tube is then expanded with a succession of mandrels of increasing size, such that the first expansion utilizes the mandrel with the smallest size and the final expansion utilizes the mandrel with the largest size. After each expansion, the laser-cut tube undergoes thermal annealing to increase its ductility as well as to reduce its hardness. The geometry in the as-cut configuration is designed such that the final annealed expanded state of the frame is the desired shape of the framework 902.

FIG. 10 shows a portion of the framework 902 shown in FIG. 9 in a compressed state, and FIG. 11 shows the same portion in an expanded state. In some examples, the second frame members 906 as seen in FIG. 10 are curved and exhibit strains at desired locations to become straighter along the length of the second frame members 906 when in the expanded state as seen in FIG. 11, whereas the first frame members 904 as seen in FIG. 10 become curved along the length of the first frame members 904 when in the expanded state as seen in FIG. 11 due to the strains placed thereon during expansion. In the example shown in FIG. 10, the second frame members 906 are curved (that is, curved while in the compressed state) and have frame projection portions 910 that extend from the second frame members 906 between the second frame member 906 and the first frame member 904. In some examples, the frame projection portions 910 extend at an angle that is substantially normal to the second frame members 906.

In some examples, it may be desirable to form the frame projection portions 910 facing the first frame members 904 that are curved in the compressed state to decrease the packing density of the framework 902, since the second frame members 906 that are curved in the compressed state achieve minimal clearance between neighboring frame members as compared to the frame member that are straight in the compressed state. Decreasing the packing density may be desirable to prevent frame members from each other before the frame achieves a crimped profile or compressed state, because contact may cause the radial compression force to increase. As the framework 902 expands as shown in FIG. 11, the second frame member 906 that were curved in the compressed state become straighter such that the frame projection portions 910 form a substantially straight line (shown by a dotted white line B-C), or are collinear with each other along the line B-C. The “straight line” or “collinear” as mentioned herein refer to a line that is substantially straight along a non-Euclidean surface, and more specifically a developable surface. Advantages in having the frame projection portions 910 align along a straight line include an improved capability for the frame projection portions 910 to restrict axial movement of the leaflet 1500, as well as allowing for the leaflet 1500 to maintain a substantially flat configuration so as to prevent undesirable distortion or wrinkling of the leaflet 1500 when the framework 902 transitions between the compressed and expanded states. Also, the straight configuration of the second frame member 906 in the expanded state minimizes wrinkling or curvature of the leaflets 1500 that may restrict opening motions thereof.

FIG. 12 compares two configurations 1200 and 1202 of the second frame members 906 that are curved in the compressed state according to some examples. In the first configuration 1200, there are two second frame members 906 that are curved in the compressed state so that a shortest distance 1204 between the two neighboring second frame members 906 is 0.45 mm. In the second configuration 1202, the second frame members 906 is curved in the compressed state and the first frame member 904 is straight in the compressed state such that a shortest distance 1206 between them is 0.61 mm. It should be noted that the distance mentioned is arbitrary, and that the shortest distances 1204 and 1206 can be any suitable length, so long as the shortest distance 1204 in the first configuration 1200 is less than the shortest distance 1206 in the second configuration 1202. In some examples, the use of frame members that are straight in the compressed configuration may result in the shortest distance 1206 to be greater than the shortest distance 1204 by 10%, 20%, 30%, 40%, 50%, 60%, 70%, 80%, 90%, or any number therebetween, with respect to the shortest distance 1204.

In some examples, the width of frame members 904, 906 may vary along the length of the frame members 904, 906. For example, an initial width 1208 of the second frame member 906 that is curved in the compressed state proximate a first base portion 1210 of the second frame member 906 is greater than a second width 1212 measured along the length of the second frame member 906 between the first base portion 1210 and a second base portion 1214. The two base portions 1210 and 1214 define the endpoints of the second frame member 906. Therefore, the second frame member 906 has a tapered configuration because the second frame member 906 has greater width proximate the two endpoints, or base portions 1210 and 1214, than at an intermediate portion between them. In some examples, one of the base portions 1210 and 1214 of the frame members 904, 906 is the intersection 908 between two or more frame members, as shown in FIG. 9. In some examples, the first frame member 904 that is straight in the compressed configuration also has a similar tapered configuration with respect to the variable width of the first frame member 904 along its length.

FIG. 13 shows the framework 902 of FIG. 9 in an expanded cylindrical configuration as previously explained according to an embodiment. The cylindrical configuration is defined by the framework 902 being positioned substantially equidistant from a central longitudinal axis Y-Y (shown with a broken line in the figure) extending through the center of the framework 902. In the expanded framework 902, the second frame members 906 that were curved in the compressed state are now straighter, and the first frame members 904 that were straight in the compressed state are now curved. Specifically, the second frame members 906 are straighter to allow the frame projection portions 910 to be aligned in a straight line along a developable surface, which in this case is the surface of a cylinder. On the other hand, the first frame members 904 that do not have the frame projection portions 910 are curved when expanded, as shown. The framework 902 in the expanded cylindrical configuration define a first end 1300 and a second end 1302. Either of the first and second ends 1300, may be the proximal end or the distal end of the framework 902 with respect to the delivery catheter 106 that is used to deploy the prosthetic valve formed using the framework 902.

Referring to FIG. 14, a framework 902 is provided in which line A-B represents a sub-cell attachment line, in this case a base attachment line, and lines B-C and C-D represent other attachment lines, for example, side and commissure attachment lines. The line B-C is disposed along the second frame members 906 that are straight in the expanded state. By having second frame members 906 that are straight in the expanded configuration along the attachment line B-C and a cell having sub-cell attachment members 206, 208 defining a sub-cell attachment line A-B, a leaflet construct (not shown) may be coupled to the framework 902 such that the base attachment region of the leaflet is positioned along the sub-cell attachment line A-B and the side attachment regions of the leaflet are positioned along, at least partially, the attachment line B-C, where the attachment line B-C is substantially straight in the expanded configuration and curved or recurved in the compressed configuration. Thus, when the framework 902 is deployed in an expanded configuration, the coupling between the leaflet construct and the framework may be such that the leaflet construct is operable to efficiently transition between a flow-blocking state and a flow-enabling state with decreased stress on the leaflet construct such as creases, unequal load bearing, and uneven coaptation.

As explained above, FIG. 15 shows the leaflet construct 1501 with a plurality of leaflets 1500 according to some embodiments in which each of the leaflets 1500 has a plurality of openings 1502 or apertures on the surface proximate an outer periphery of the leaflet 1500 called a base attachment region 1504. The base attachment region 1504 receives and holds the frame projection portions 910. In various embodiments, leaflet free edges 1506 of adjacently situated leaflets 1500 come together at a coaptation region. In some embodiments, the leaflets 1500 meet or nearly meet at a triple point 1508 such that the leaflets 1500 come together in a Y-shaped pattern when viewed from above.

FIG. 16 shows the leaflet 1500 with the frame projection portion 212 extending through the openings 1502 such that the frame projection portion 212 provides a mechanical interference to impede the base attachment region 1504 of the leaflet 1500 decoupling from the frame projection portion 212 as previously explained. In some examples, the frame projection portion 212 may have different shapes, including hooks, barbs, zippers, clips, clasps, coils, or any other suitable configuration.

It is contemplated within the scope of this disclosure that the features described herein may be implemented on a variety frames including those discussed herein, an inner or an outer frame (e.g., nesting frame arrangements), or a multi-part frame arrangement.

### Frame Materials

It is appreciated that the frame can be formed via various manufacturing processes, including, but not limited to, additive manufacturing, subtractive manufacturing, and injection molding. For example the frame may be formed of wire or braided wire, formed by three-dimensional printing, as well as formed by etching, cutting, laser cutting, and stamping sheets or tubes of material, among other suitable processes, into an annular or tubular structure or, if a sheet of material, with the sheet then formed into an annular or tubular structure. In various examples, the frame can comprise, such as, but not limited to, any biocompatible and, in those embodiments where applicable, elastically deformable metallic or polymeric material including shape-memory materials, such as nitinol, a nickel-titanium alloy. Other materials suitable for the frame include, but are not limited to, other titanium alloys, stainless steel, biocompatible alloys (e.g., cobalt-chromium alloy, cobalt-nickel alloy, nickel-cobalt-chromium alloy, or nickel-cobalt-chromium-molybdenum alloy, such as MP35N® alloy (SPS Technologies, Jenkintown, Pa.)), polymers, polypropylene, polyethylene terephthalate, PEEK, acetyl homopolymer, acetyl copolymer, other alloys, polymers, and thermoplastics, or any other material that is generally biocompatible having adequate physical and mechanical properties to function as a frame as described herein, or combinations thereof.

It is understood that when the frame is constructed of a plastically-deformable material, the frame, and thus the prosthetic valve, can have a lower radial dimension when coupled to the delivery catheter and then can be expanded to a larger radial dimension inside a patient, either by internal forces or by external forces, such as by an inflatable balloon or equivalent expansion mechanism. When constructed of an elastic material, the frame, and thus the prosthetic valve, can be crimped to a radially collapsed configuration and restrained in the collapsed configuration by, for example, insertion into a sheath or constrained by fibers or other constraining mechanism. Once inside the body, the prosthetic valve can be released from the constraining mechanism, which allows the prosthetic valve to expand to its functional size.

### Leaflet Materials

For simplicity of discussion, when referring to a material from which a leaflet is made, it is appreciated that the same material may also be used to make a leaflet construct. Therefore, in this context, the term “leaflet” will refer to both leaflet and leaflet construct. It is understood that in embodiments presented herein, the leaflet is flexible and is comprised of a flexible material.

In some examples the leaflet is formed of a natural material, such as repurposed tissue, including bovine tissue, porcine tissue, or the like. In other examples, the leaflet is formed from a synthetic material, such as a biocompatible polymer.

Examples of suitable biocompatible polymers for use in making a synthetic flexible leaflet include, but are not limited to, the groups including urethanes, silicones (e.g., organopolysiloxanes), copolymers of silicon-urethane, styrene/isobutylene copolymers, polyisobutylene, polyethylene, polyethylene-co-poly(vinyl acetate), polyester copolymers, nylon copolymers, fluorinated hydrocarbon polymers and copolymers and/or mixtures of each of the foregoing, and composite materials made therewith. Examples of a suitable fluoroelastomers include, but are not limited to, copolymers of tetrafluoroethylene and perfluoromethyl vinyl ether (TFE/PMVE copolymer) and (per)fluoroalkylvinylethers (PAVE). Such polymers may exhibit the physical properties of an elastomer, elastomeric, or non-elastomeric material.

Examples of processes to form a synthetic leaflet include, but are not limited to, casting, injection molding, extrusion, and imbibing. In accordance with some embodiments, the leaflet is a composite material that includes at least one membrane as defined above and discussed below combined with a polymer. The polymer may be a coating or layer on the membrane and/or may be imbibed into a microporous structure of the membrane. Examples of a membrane include, but not limited to, microporous polyethylene and expanded fluoropolymer membrane such as expanded polytetrafluoroethylene (ePTFE). The expanded fluoropolymer membrane used to form some of the composite materials described can comprise PTFE homopolymer, blends of PTFE, expandable modified PTFE and/or expanded copolymers of PTFE. As an example of a microporous membrane, ePTFE comprises a matrix of fibrils defining a plurality of spaces within the matrix. The polymer may be imbibed or otherwise incorporated into the plurality of spaces to form the composite material.

It is appreciated that multiple types of membranes and multiple types of polymer can be combined to form a composite material while remaining within the spirit and scope of the present disclosure. Additional materials may be incorporated into the polymer, such as, but not limited to, inorganic fillers, therapeutic agents, radiopaque markers, and the like while remaining within the spirit and scope of the present disclosure. In accordance with some examples, the composite material comprises porous synthetic polymer membrane by weight in a range of about 10% to about 90%.

By way of example of an elastomer, TFE/PMVE copolymer is an elastomer when comprising essentially of between 60 and 20 weight percent tetrafluoroethylene and respectively between 40 and 80 weight percent perfluoromethyl vinyl ether. By way of example of an elastomeric material TFE/PMVE copolymer is an elastomeric material when comprising essentially of between 67 and 61 weight percent tetrafluoroethylene and respectively between 33 and 39 weight percent perfluoromethyl vinyl ether. By way of example of a non-elastomeric material, TFE/PMVE copolymer is a non-elastomeric material when comprising essentially of between 73 and 68 weight percent tetrafluoroethylene and respectively between 27 and 32 weight percent perfluoromethyl vinyl ether. The TFE and PMVE components of the TFE-PMVE copolymer are presented in wt %. For reference, the wt % of PMVE of 40, 33-39, and 27-32 corresponds to a mol % of 29, 23-28, and 18-22, respectively. The TFE-PMVE copolymer exhibits elastomer, elastomeric, and/or non-elastomeric material properties based on the wt % or mol % of the respective polymers.

In accordance with some embodiments herein, the leaflet comprises a composite material having at least one porous synthetic polymer membrane layer having a plurality of pores and/or spaces and a polymer that is an elastomer and/or an elastomeric material filling the pores and/or spaces of the at least one synthetic polymer membrane layer. In accordance with other examples, the leaflet further comprises a layer or coating of an elastomer and/or an elastomeric material and/or a non-elastomeric material on one or both sides of the composite material. In some examples the leaflet is an expanded polytetrafluoroethylene (ePTFE) membrane having been imbibed with TFE-PMVE copolymer comprising from about 60 to about 20 weight percent tetrafluoroethylene and respectively from about 40 to about 80 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces. In other examples the leaflet is an ePTFE membrane having been imbibed with TFE-PMVE copolymer comprising from about 70 to about 61 weight percent tetrafluoroethylene and respectively from about 33 to about 39 weight percent perfluoromethyl vinyl ether, the leaflet further including a coating of TFE-PMVE copolymer comprising from about 73 to about 68 weight percent tetrafluoroethylene and respectively about 27 to about 32 weight percent perfluoromethyl vinyl ether on the blood-contacting surfaces.

### Tissue Ingrowth

In various embodiments, the leaflet 1500 is constructed in a manner that promotes tissue ingrowth. In some embodiments, the leaflet 1500 may be constructed to encourage tissue ingrowth and proliferation across one or more discrete regions, portions, or sections of one or more of the materials forming the leaflet 1500, or alternatively across an entirety of one or more of the materials forming the leaflet 1500. Tissue ingrowth and proliferation may be promoted on an outflow side or surface of the leaflet 1500, and/or on an inflow side or surface of the leaflet [999], and/or within one or more materials forming the leaflet.

In various embodiments, the leaflets 1500 include a composite material combined with a tissue ingrowth curtain that may be incorporated into composite material and/or coupled to the composite material.

In various embodiments, one or more portions of the leaflet frame 108 may be covered with material suitable for promoting tissue ingrowth. For example, the leaflet frame 108 can be wrapped with a material, suitable for promoting tissue ingrowth. In various examples, such tissue ingrowth promoting materials can be applied to leaflet frame 108 entirely, or alternatively to less than all of the leaflet frame 108. For example, suitable materials for promoting tissue ingrowth could be coupled to the leaflet frame inner surface and the leaflet frame outer surface of the leaflet frame. Some nonlimiting examples of materials that can be applied to the leaflet frame 108 (or other portions of the prosthetic valve 100) include expanded polytetrafluoroethylene (ePTFE), such as an ePTFE membrane, as well as fabric, film, or coating, and a polyethylene terephthalate fabric (e.g., Dacron fabric).

According to some examples, as will be discussed in greater detail below, this promotion of tissue ingrowth is facilitated by the coupling of one or more synthetic tissue ingrowth curtains to one or more composite materials such that tissue is encouraged to grow (or is not otherwise prevented or inhibited from growing) into and/or onto the one or more tissue ingrowth curtains. That is, in some examples, one or more layers configured to promote tissue ingrowth may be applied to the composite material. In some examples, as described herein, the underlying leaflet structure or material may be configured to inhibit or prevent tissue ingrowth.

Additionally or alternatively, in some examples, this promotion of tissue ingrowth is facilitated by selectively imbibing, such as with one or more fluoroelastomers, one or more portions of the one or more materials forming the leaflet 1500. Reference to “selectively imbibing” is referring to the act of imbibing a porous material with a filling material at selected portions of the porous material or to a lesser degree leaving a degree of porosity of the porous material.

That is, in some examples, in addition to or as an alternative to coupling one or more synthetic tissue ingrowth curtains to one or more composite materials, the composite material as discussed above regarding leaflet materials is configured to promote or accommodate tissue ingrowth. In some such examples, as discussed in greater detail below, the composite material is configured such that tissue is encouraged to grow (or is not otherwise prevented or inhibited from growing) into and/or onto one or more discrete or designated sections, portions, or regions of the composite material by way of selectively imbibing the membrane associated with those portions.

In various embodiments, the tissue ingrowth curtain generally includes an expanded fluoropolymer membrane, which comprises a plurality of spaces within a matrix of fibrils that is suitable for promoting and supporting the ingrowth of tissue. Other nonlimiting example materials include other biocompatible porous materials such as porous polyethylene membrane and knit PTFE. However, as mentioned above, and as discussed in greater detail below, in some examples the tissue ingrowth curtain(s) may be applied to the composite material in the form of one or more coatings.

In some examples, the tissue ingrowth curtain includes an expanded fluoropolymer made from a porous ePTFE membrane. However, it is appreciated that the tissue ingrowth curtain may be formed from a number of different types of membranes, including other fluoropolymer membranes, and other biocompatible porous materials such as porous polyethylene membrane and knit PTFE. For instance, the expandable fluoropolymer can comprise PTFE homopolymer. In some examples, the tissue ingrowth curtain can be formed from copolymers of hexafluoropropylene and tetrafluoroethylene, such as Fluorinated Ethylene Propylene (FEP). In some examples, blends of PTFE, expandable modified PTFE and/or expanded copolymers of PTFE can be used. It will thus be appreciated that the tissue ingrowth curtain may be formed from a variety of different polymeric materials, provided they are biocompatible and possess or are modified to include a suitable microstructure suitable for promoting or supporting tissue ingrowth. In various examples, the tissue ingrowth curtains may range in thickness from between one microns and four hundred microns depending on the selected material.

In some examples, the polymeric material may include one or more naturally occurring and/or one or more artificially created pores, reliefs, channels, and/or predetermined surface topology, suitable for supporting tissue ingrowth. Other biocompatible materials which can be suitable for use forming the tissue ingrowth curtain include but are not limited to the groups of urethanes, fluoropolymers, styrene/isobutylene copolymers, polyisobutylene, polyethylene-co-poly(vinyl acetate), polyester copolymers, nylon copolymers, fluorinated hydrocarbon polymers and copolymers or mixtures of each of the foregoing.

While the above-discussed tissue ingrowth curtains generally include membranes, films, knits, or other structures that are bonded, applied, or otherwise attached to the composite material, as mentioned above, in some examples the tissue ingrowth curtain(s) may be applied to the composite material in the form of one or more coatings. In some such example, a coherent irregular network is distributed or deposited onto one or more portions, regions, sections, areas, or zones of the composite material. In some examples, the coherent irregular network is applied to one or more portions of the composite material to create a surface texture suitable for supporting the ingrowth and proliferation of tissue, as those of skill will appreciate. For example, the coherent irregular network may be selectively applied to one or more discrete or designated sections, portions, or regions of the composite material. In some such examples, the coherent irregular network is applied to the designated areas by masking or otherwise covering those portions of the underlying leaflet where ingrowth of tissue is undesirable such that the cover or mask can be removed subsequent to the coherent irregular network application process to achieve a leaflet having a first region including the coherent irregular network and a second region free of a coherent irregular network. In some examples, one or more sacrificial sheets, such as one or more polyimide sheets (e.g., Kapton sheets), are arranged on the composite material and operate to mask or otherwise prevent the coherent irregular network from being applied to the masked or covered areas. Some nonlimiting examples of sacrificial sheet materials include polyester, polyetheretherketone (PEEK), PET, ePTFE/Kapton blends such as mapton, ePTFE, PTFE, silicones, and stainless steel, or other thin metal sheeting. In some examples, the one or more sacrificial sheets can be removed after the coherent irregular network application process to reveal a leaflet having a structure including one or more regions including the coherent irregular network and one or more regions free of the coherent irregular network (e.g., where the underlying composite material is exposed). Such a configuration provides for a construction of the leaflet that minimizes a possibility for delamination between bonded membrane layers.

As mentioned above, in some examples, in addition to or as an alternative to applying one or more tissue ingrowth curtains to the composite material, the composite material is configured to promote or accommodate tissue ingrowth. For instance, in some examples, the composite material is configured such that tissue is encouraged to grow (or is not otherwise prevented or inhibited from growing) into and/or onto one or more discrete or designated sections, portions, or regions of the composite material. For instance, as mentioned above, the composite material forming the synthetic leaflet may include an elastomer and/or an elastomeric material such as a fluoroelastomer imbibed or otherwise incorporated into the expanded fluoropolymer membrane. In various examples, to achieve a composite material that promotes or otherwise accommodates the ingrowth and proliferation of tissue the expanded fluoropolymer membrane is selectively imbibed, such as with one or more fluoroelastomers, such that the expanded fluoropolymer membrane includes one or more discrete portions, regions, sections, zones, or areas that are free of or are not otherwise imbibed with the elastomeric filler material (or at least are not filled to the extent that the elastomeric filler material operates to prevent tissue ingrowth). Selectively imbibing the membrane of the composite material may be done in accordance with techniques as known to those of skill in the art.

While the above discussed embodiments and examples include applying a tissue ingrowth curtain to one or more portions of one or more surfaces of the composite material, or selectively imbibing one or more portions of one or more sides of a membrane of the composite material with a filler material, it is appreciated that, in various examples, a leaflet may be constructed by both imbibing one or more portions of the membrane and applying a tissue ingrowth curtain to the selectively imbibed membrane.

In various examples, the membrane may be imbibed with a plurality of filler materials. That is, in some examples, a first portion, area, region, section, or zone of the membrane of composite material may be imbibed with a first filler material while a second portion, area, region, section, or zone of the membrane of the composite material is imbibed with a second filler material. For instance, in some examples, a first portion of the membrane of the composite material is imbibed with a first filler material such that the first portion of the membrane is resistant to or otherwise inhibits or prevents tissue ingrowth into and/or onto and/or across the first portion. However, in some examples, those portions of the membrane imbibed with the first filler may also be unsuitable for accommodating the bonding or coupling of a tissue ingrowth curtain. Accordingly, in examples where it is desirable bond or otherwise couple a tissue ingrowth leaflet to a second portion of the membrane, the second portion may be imbibed with a second filler material such that the second portion of the membrane is suited to have a tissue ingrowth curtain bonded or otherwise coupled thereto. In some examples, the second filler material may additionally or alternatively encourage tissue ingrowth. That is, in some examples, one or more portions of the membrane may be imbibed with a filler material that encourages tissue ingrowth and proliferation. Alternatively, as mentioned above, the second portion may not be imbibed with any filler material at all, but may instead remain free of filler material.

In some examples, the method includes applying an adhesive to the membrane in addition to or as an alternative to applying the adhesive to the tissue ingrowth curtain, as discussed above. In some examples, an adhesive, such as FEP, is similarly wicked or imbibed into one or more portions of the membrane, after which the tissue ingrowth curtain and the membrane are pressed together and/or heat set according to known methods.

In some other examples, in addition to or as an alternative to applying adhesives to the tissue ingrowth curtain and the membrane separately or individually, the tissue ingrowth curtain (e.g., having a designated pattern) and the membrane are layered with one or more adhesives or adhesive layers therebetween, after which the layered construct is pressed and/or heat set according to known methods. The method further includes cutting the leaflet from the resulting construct according to known methods. In some examples, a final free edge cutting operation may be performed on the leaflet to achieve a clean free edge of the resulting leaflet according to known methods, as those of skill will appreciate.

In accordance with an embodiment, the composite material can include an expanded fluoropolymer made from porous ePTFE membrane.

The expanded fluoropolymer membrane, used to form some of the composites described, can comprise PTFE homopolymer. In alternative embodiments, blends of PTFE, expandable modified PTFE and/or expanded copolymers of PTFE can be used.

### Bio-Active Agents

In some embodiment, all or a part of the prosthetic valve, including the leaflets, may be provided with a biologically active (bio-active) agent. Bio-active agents can be coated onto a portion or the entirety of the prosthetic valve, including the leaflet and/or leaflet construct, for controlled release of the agents once the prosthetic valve is implanted. The bio-active agents can include, but are not limited to, vasodilator, anti-coagulants, anti-platelet, anti-thrombogenic agents such as, but not limited to, heparin. Other bio-active agents can also include, but are not limited to agents such as, for example, anti-proliferative/antimitotic agents including natural products such as vinca alkaloids (e.g., vinblastine, vincristine, and vinorelbine), paclitaxel, epidipodophyllotoxins (e.g., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (e.g., estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives e.g., aspirin; para-aminophenol derivatives e.g., acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligonucleotides and combinations thereof; cell cycle inhibitors, mTOR inhibitors, and growth factor receptor signal transduction kinase inhibitors; retinoids; cyclin/CDK inhibitors; HMG co-enzyme reductase inhibitors (statins); and protease inhibitors.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A prosthetic valve comprising:
a leaflet frame configured to be transitioned from a collapsed state having a first diameter to an expanded state having a second diameter that is larger than the first diameter, the leaflet frame having an inlet end and an outlet end and defining a longitudinal axis, the leaflet frame including,
a plurality of frame members that form a framework defining plurality of cells each having an interior cell space, the plurality of cells including at least one attachment cell, the plurality of frame members including a plurality of sub-cell attachment members extending into the interior cell space of the at least one attachment cell, the sub-cell attachment members each being configured to define a plurality of attachment locations along a sub-cell attachment line that is defined within a plane that intersects with, and is optionally transverse to, the longitudinal axis such that the sub-cell attachment line extends through the interior cell space of the at least one attachment cell.

2. The prosthetic valve of claim 1, further comprising a leaflet construct including a leaflet having a base attachment region, wherein the base attachment region of the leaflet is coupled to the leaflet frame along the sub-cell attachment line.

3. The prosthetic valve of claim 1, wherein each of the plurality of sub-cell attachment members includes a projection base portion extending into the interior cell space and a projection head portion opposite the projection base portion, each of the projection head portions of the plurality of sub-cell attachment members being positioned along the sub-cell attachment line.

4. The prosthetic valve of claim 1, wherein the plurality of sub-cell attachment members includes a first sub-cell attachment member and a second sub-cell attachment member, and further wherein the at least one attachment cell has a top cell portion defined by the framework and a bottom cell portion defined by the framework, the first sub-cell attachment member extending from the top cell portion and the second sub-cell attachment member extending from the bottom cell portion.

5. The prosthetic valve of claim 4, wherein the first and second sub-cell attachment members are substantially parallel to each other when the leaflet frame is in the expanded state.

6. The prosthetic valve of claim 4, wherein the top cell portion is defined by a first frame member and a second frame member of the plurality of frame members, and further wherein the first sub-cell attachment member includes a first leg extending from the first frame member and a second leg extending from second frame member, the first and second legs intersecting to form a first sub-cell attachment joint, the first sub-cell attachment joint being positioned adjacent the sub-cell attachment line when the leaflet frame is in the expanded state.

7. The prosthetic valve of claim 4, wherein the bottom cell portion is defined by a third frame member and a fourth frame member of the plurality of frame members, and further wherein the second sub-cell attachment member includes a third leg extending from the third frame member and a fourth leg extending from the fourth frame member, the third and fourth legs intersecting to form a second sub-cell attachment joint, the second sub-cell attachment joint being positioned adjacent the sub-cell attachment line when the leaflet frame is in the expanded state.

8. The prosthetic valve of claim 4, wherein the first sub-cell attachment member and the framework together define a shape resembling a parallelogram when the leaflet frame is in the expanded state, and optionally, wherein the second sub-cell attachment member and the framework combine to define a shape resembling a parallelogram when the leaflet frame is in the expanded state.

9. The prosthetic valve of claim 6, wherein the first leg of the first sub-cell attachment member is shorter than the second leg of the first sub-cell attachment member, and optionally, wherein the first leg of the first sub-cell attachment member is thinner than the second leg of the first sub-cell attachment member.

10. The prosthetic valve of claim 7, wherein the third leg of the second sub-cell attachment member has substantially the same length and width as the first leg of the first sub-cell attachment member, and optionally, wherein the fourth leg of the second sub-cell attachment member has substantially the same length and width as the second leg of the first sub-cell attachment member.

11. The prosthetic valve of claim 1, wherein the plurality of frame members include a plurality of leaflet attachment members configured to be coupled to first and second sides of one or more leaflets of a leaflet construct, each of the plurality of leaflet attachment members having a first end portion at which each of the plurality of leaflet attachment members intersects with another one of the plurality of frame members, a second end portion at which each of the plurality of leaflet attachment members intersects with another one of the plurality of frame members, and an intermediate portion between the first and second end portions, the intermediate portion of each of the plurality of leaflet attachment members having a first curvature when the leaflet frame is in the collapsed state and a second curvature when the leaflet frame is in the expanded state, the first curvature being greater than the second curvature.

12. The prosthetic valve of claim 11, wherein the first curvature is defined as a maximum transverse deviation of the intermediate portion from a straight line drawn between the first and second end portions, the maximum transverse deviation having a value of 0.5 (1/mm).

13. The prosthetic valve of claim 11, wherein the second curvature is defined as a maximum transverse deviation of the intermediate portion from a straight line drawn between the first and second end portions, the maximum transverse deviation having a value of 0.05 (1/mm).

14. The prosthetic valve of claim 11, wherein the second curvature is at least 20% less than the first curvature.

15. The prosthetic valve of claim 11, wherein the intermediate portions of at least two of the leaflet attachment members are linearly aligned with one another when the leaflet frame is in the expanded state.

16. The prosthetic valve of claim 11, wherein the intermediate portions of each of the leaflet attachment members has a recurved shape when the leaflet frame is in the collapsed state.

17. The prosthetic valve of claim 11, further comprising a leaflet construct including a leaflet having a side attachment region, the side attachment region being coupled to at least one of the plurality of leaflet attachment members, and optionally, wherein the side attachment region is coupled to at least two of the plurality of leaflet attachment members having intermediate portions that are in linear alignment when the leaflet frame is in the expanded state.

18. A prosthetic valve comprising:
a leaflet frame configured to be transitioned from a collapsed state having a first diameter to an expanded state having a second diameter that is larger than the first diameter, the leaflet frame having an inlet end and an outlet end and defining a longitudinal axis, the leaflet frame including,
a plurality of frame members defining a framework forming a pattern of cells, the plurality of frame members including a plurality of leaflet attachment members configured to be coupled to first and second sides of one or more leaflets of a leaflet construct, each of the plurality of leaflet attachment members having a first end portion at which each of the plurality of leaflet attachment members intersects with another one of the plurality of frame members, a second end portion at which each of the plurality of leaflet attachment members intersects with another one of the plurality of frame members, and an intermediate portion between the first and second end portions, the intermediate portion of each of the plurality of leaflet attachment members having a first curvature when the leaflet frame is in the collapsed state and a second curvature when the leaflet frame is in the expanded state, the first curvature being greater than the second curvature.

19. The prosthetic valve of claim 18, wherein the first curvature is defined as a maximum transverse deviation of the intermediate portion from a straight line drawn between the first and second end portions, the maximum transverse deviation having a value of 0.5 (1/mm).

20. The prosthetic valve of claim 18, wherein the second curvature is defined as a maximum transverse deviation of the intermediate portion from a straight line drawn between the first and second end portions, the maximum transverse deviation having a value of 0.05 (1/mm).

21. The prosthetic valve of claim 18, wherein the second curvature is at least 20% less than the first curvature.

22. The prosthetic valve of claim 18, wherein the intermediate portions of at least two of the leaflet attachment members are linearly aligned with one another when the leaflet frame is in the expanded state.

23. The prosthetic valve of claim 18, wherein the intermediate portions of each of the leaflet attachment members has a recurved shape when the leaflet frame is in the collapsed state.

24. The prosthetic valve of claim 18, further comprising a leaflet construct including a leaflet having a side attachment region, the side attachment region being coupled to at least one of the plurality of leaflet attachment members, and optionally, wherein the side attachment region is coupled to at least two of the plurality of leaflet attachment members having intermediate portions that are in linear alignment when the leaflet frame is in the expanded state.

25. A method for treating a human patient with a diagnosed condition or disease associated with valve insufficiency or valve failure of a native valve, the method comprising implanting the prosthetic valve of claim 1, the prosthetic valve being implanted at or adjacent to a location associated with the native valve.
